(19) United States
(12) Patent Application Publication
(10) Pub. No.: US 2021/0033638 A1
(43) Pub. Date: Feb. 4, 2021

(54) MOTION SENSING MODULE

(71) Applicants: Yen-Chi Lee, New Taipei City (TW); Fu-Te Yuan, New Taipei City (TW)

(72) Inventors: Yen-Chi Lee, New Taipei City (TW); Fu-Te Yuan, New Taipei City (TW)

(73) Assignee: iSentek Inc., New Taipei City (TW)

(21) Appl. No.: 16/944,109

(22) Filed: Jul. 30, 2020

Related U.S. Application Data
(60) Provisional application No. 62/880,652, filed on Jul. 31, 2019.

(51) Int. Cl. G01P 13/00; G01D 5/16

(52) U.S. Cl. CPC G01P 13/00; G01D 5/16

(57) ABSTRACT

A motion sensing module including a plurality of first and second magneto-resistive sensors and a processor is provided. The processor executes the following steps S1 and S2. The step S1: the processor defines at least one first coordinate system from a first portion of the first magneto-resistive sensors and a second portion of the second magneto-resistive sensors. The processor defines at least one second coordinate system from a third portion of the first magneto-resistive sensors and a fourth portion of the second magneto-resistive sensors. The first and the second coordinate systems are rotational symmetry to each other. The step S2: the first and second magneto-resistive sensors generate a plurality of sensing results according to an external magnetic field. The processor performs calculations according to these sensing results based on the first and second coordinate systems to obtain a calculation result and measures motion information according to the calculation result.

### CROSS-REFERENCE TO RELATED APPLICATION

This application claims the priority benefit of U.S. provisional application No. 62/880,652, filed on Jul. 2019The entirety of the above-mentioned patent application is hereby incorporated by reference herein and made a part of this specification.

### TECHNICAL FIELD

The invention relates to a motion sensing module, and more particularly, to a motion sensing module having a magneto-resistive sensor.

### BACKGROUND

With the advancement of technology, a motion sensor for detecting objects are widely used in different fields such as Virtual Reality (VR), Augmented Reality (AR), drones or smart homes. Optical motion sensors have the advantages of high precision and fast speed; however, they can be easily affected by ambient light, dust in the air and object, and have higher cost. Inertial motion sensors have advantages of fast response, satisfactory precision, and low cost, but can be affected by ambient magnetic field. Global Positioning System (GPS) is currently only used outdoors so its application is limited.

Accordingly, motion sensors that use magnetic sensors to detect an object motion state have been widely used in recent years to avoid the above problems. The main principle is to determine an object velocity or an object position by a variation of magnetic field with respect to time and a corresponding calculation method. In general, the function of magnetic field with respect to time is a continuous smooth curve. If directions of differential results obtained by a differential operation of the magnetic field with respect to time before and after a curve turning point are not the same, calculated velocity values will be sharply going upward and downward at certain moments. This phenomenon will cause serious errors in subsequent determinations for the object velocity and the object position.

### SUMMARY

The invention provides a motion sensing device having a favorable sensing capability.

The motion sensing device of an embodiment of the invention is suitable for being mounted on a to-be-measured object and used for sensing motion of the to-be-measured object. The to-be-measured object being placed within a magnetic field range of an external magnetic field. The motion sensing module includes a plurality of first magneto-resistive sensors, a plurality of second magneto-resistive sensors and a processor. The first magneto-resistive sensors are disposed on a first reference plane. The second magneto-resistive sensors are disposed on a second reference plane. The first reference plane is different from the second reference plane and parallel to the second reference plane. Positions of the first magneto-resistive sensors correspond to positions of the second magneto-resistive sensors, respectively. The processor is coupled to the first magneto-resistive sensors and the second magneto-resistive sensors. The processor divides the first magneto-resistive sensors into a first portion and a third portion different from each other and divides the second magneto-resistive sensors into a second portion and a fourth portion different from each other. The processor executes the following steps. The step S1: the processor defines at least one first coordinate system from a first portion of the first magneto-resistive sensors and a second portion of the second magneto-resistive sensors. The processor defines at least one second coordinate system from a third portion of the first magneto-resistive sensors and a fourth portion of the second magneto-resistive sensors. The first and the second coordinate systems are rotational symmetry to each other. The step S2: the first magneto-resistive sensors and the second magneto-resistive sensors generate a plurality of sensing results according to an external magnetic field, and the processor performs calculations according to the sensing results based on the first coordinate system and the second coordinate system to obtain a calculation result and measures motion information according to the calculation result.

In an embodiment of the invention, the processor further executes the following steps. The step S3: the step S1 and the step S2 repeated to obtain calculation results corresponding to other first coordinate systems and other second coordinate systems. The step S4: at least a portion of all the calculation results is obtained and averaged to measure the motion information.

In an embodiment of the invention, the motion information is a velocity of the to-be-measured object.

In an embodiment of the invention, in the step S2, the processor performs the calculations according to the sensing results based on the first coordinate system and the second coordinate system to measure the velocity of the to-be-measured object by an equation:

$${\to }_{V} = {J ( {\to }_{B} )}^{- 1} \times \frac{{d}_{{\to }_{ B }}}{dt}$$

wherein

$${->}_{V}$$

is the velocity of the to-be-measured object,

$${J ( {\to }_{B} )}^{- 1}$$

is an inverse matrix of a matrix obtained by the processor after performing a Jacobian matrix operation according to the sensing results based on the first coordinate system and the second coordinate system, and

$$\frac{{d}_{{\to }_{ B }}}{dt}$$

is a differential operation of the sensing results with respect to time.

In an embodiment of the invention, after integrating the velocity of the to-be-measured object with respect to time, the processor obtains position information of the to-be-measured object at a specific time according to an initial position of the to-be-measured object.

In an embodiment of the invention, the processor uses one of the first magneto-resistive sensors in the first portion as a coordinate origin magneto-resistive sensor, and uses two of the first magneto-resistive sensors adjacent to the coordinate origin magneto-resistive sensor in the first portion and one of the second magneto-resistive sensors corresponding to the coordinate origin magneto-resistive sensor as coordinate direction magneto-resistive sensors. A vector from the coordinate origin magneto-resistive sensor to one of the coordinate direction magneto-resistive sensors is defined as a direction vector of the first coordinate system.

In an embodiment of the invention, the processor uses one of the second magneto-resistive sensors in the second portion as a coordinate origin magneto-resistive sensor, and uses two of the second magneto-resistive sensors adjacent to the coordinate origin magneto-resistive sensor in the second portion and one of the first magneto-resistive sensors corresponding to the coordinate origin magneto-resistive sensor as coordinate direction magneto-resistive sensors.

A vector from the coordinate origin magneto-resistive sensor to one of the coordinate direction magneto-resistive sensors is defined as a direction vector of the second coordinate system.

In an embodiment of the invention, the positions of the first magneto-resistive sensors are aligned with the positions of the second magneto-resistive sensors in a one to one manner.

In an embodiment of the invention, the first portion and the second portion are rotational symmetry to each other, and the third portion and the fourth portion are rotational symmetry to each other.

Based on the above, according to the motion sensing device in the embodiments of the invention, the processor defines the first and the second coordinate systems which are rotational symmetry to each other for the first and the second magneto-resistive sensors disposed on the different reference planes, and performs the calculations according to the sensing results sensed from the external magnetic field by the magneto-resistive sensors based on the first and the second coordinate systems. The directions of the calculation results obtained before and after certain moments are opposite if only one of the first and the second coordinate system is used. In the embodiments of the invention, by taking both the calculation results of the first and the second coordinate systems into account, the motion sensing device can eliminate the errors derived during the process of the calculations, and thus, can accurately measure the motion information of the to-be-measured object.

### BRIEF DESCRIPTION OF THE DRAWINGS

FIG. 1 is a schematic diagram of a motion sensing device mounted on a to-be-measured object according to an embodiment of the invention.

FIG. 2A is a block diagram of the motion sensing device in FIG. 1.

FIG. 2B is a schematic diagram illustrating architecture of multiple magnetoresistive sensors of the motion sensing device in FIG. 1.

FIG. 3A to FIG. 3D respectively show different first coordinate systems and different second coordinate systems.

FIG. 4A shows an equation of earth magnetic field underwent a Jacobian matrix operation.

FIG. 4B shows a differential equation of earth magnetic field with respect to time.

FIG. 5A shows a velocity of the to-be-measured object calculated by the processor according to the sensing results sensed by the magnetoresistive sensors only based on the first coordinate system.

FIG. 5B shows a velocity of the to-be-measured object measured by the processor according to the sensing results sensed by the magnetoresistive sensors only based on the second coordinate system.

FIG. 5C shows a velocity of the to-be-measured object measured by the processor according to the sensing results sensed by the magnetoresistive sensors based on the first and the second coordinate systems.

### DETAILED DESCRIPTION

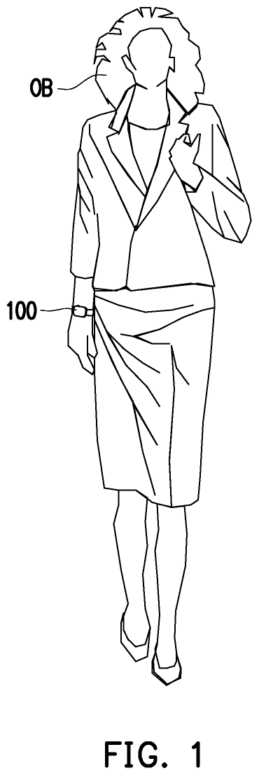

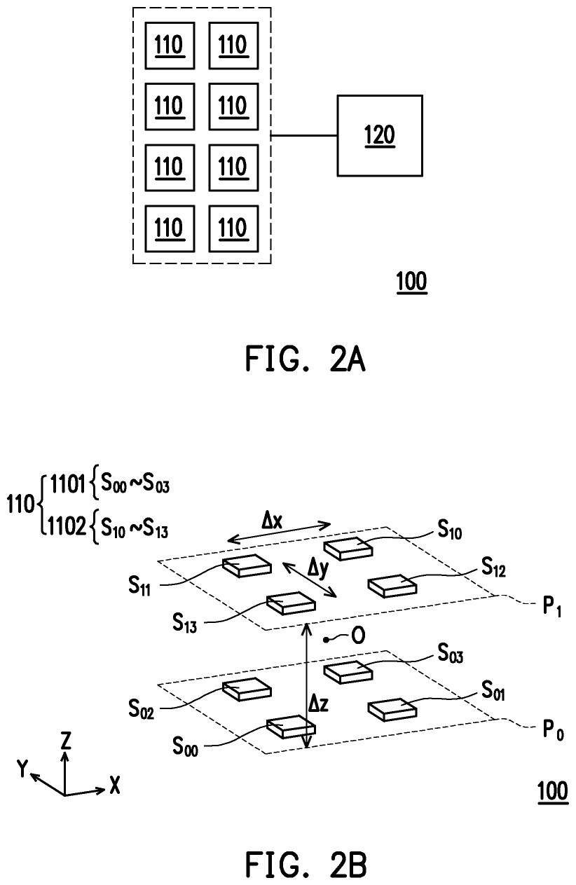

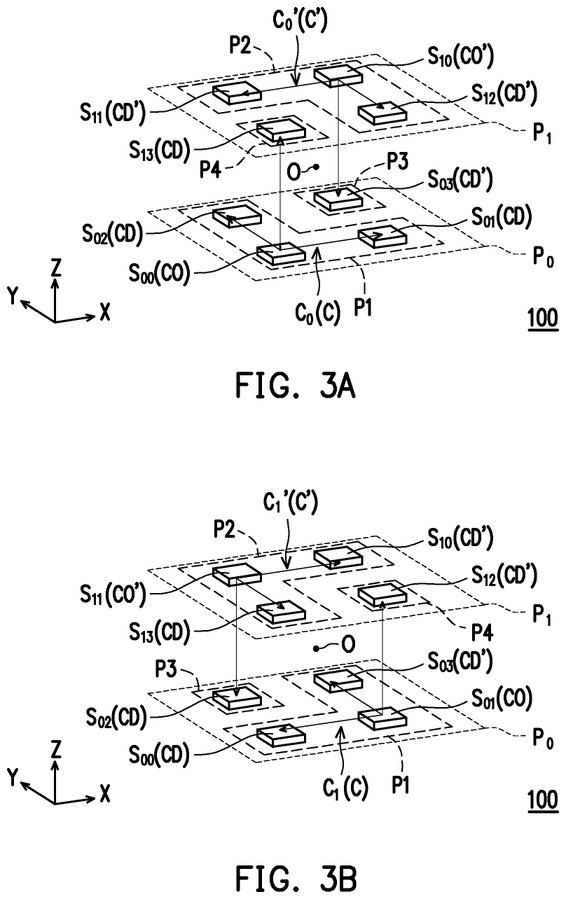

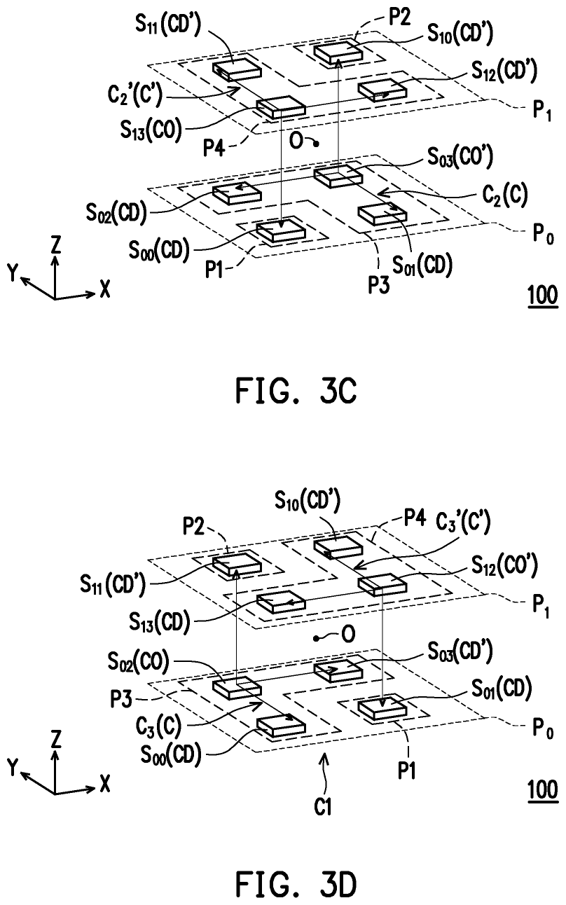

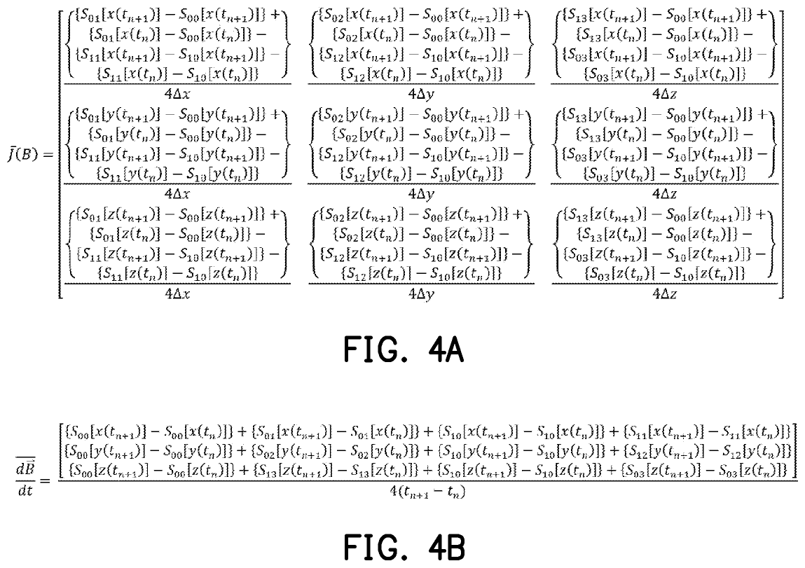

FIG. 1 is a schematic diagram of a motion sensing device mounted on a to-be-measured object according to an embodiment of the invention. FIG. 2A is a block diagram of the motion sensing device in FIG. 1. FIG. 2B is a schematic diagram illustrating architecture of multiple magnetoresistive sensors of the motion sensing device in FIG. 1. FIG. 3A to FIG. 3D respectively show different first coordinate systems and different second coordinate systems. FIG. 4A shows an equation of earth magnetic field underwent a Jacobian matrix operation. FIG. 4B shows a differential equation of earth magnetic field with respect to time.

For ease of description, a motion sensing device 100 of this embodiment can be regarded as being placed within a three-dimensional space formed by X-axis, Y-axis and Z-axis, which are perpendicular to each other.

Referring to FIG. 1, in this embodiment, the motion sensing device 100 is suitable for being mounted on a to-be-measured object OB and used for sensing motion of the to-be-measured object OB. Here, the to-be-measured object OB is, for example, human, but not limited thereto. Further, the to-be-measured object OB is placed within a magnetic field range of an external magnetic field. Here, the external magnetic field is, for example, earth magnetic field (not shown), but not limited thereto. Referring to FIG. 2A and FIG. 2B, the motion sensing device 100 includes a plurality of magneto-resistive sensors 110 and a processor 120. In the following paragraphs, the above-mentioned components and their corresponding configuration relationships will be described in detail.

The magneto-resistive sensor 110 refers to a sensor whose resistance can be changed correspondingly through changes in the external magnetic field. Types of the magneto-resistive sensors 110 include anisotropic magneto-resistive sensors, tunneling magneto-resistive sensors, giant magneto-resistive sensors, or flux gates, but are not limited thereto. In this embodiment, for example, there are eight magneto-resistive sensors 110, respectively disposed on reference planes P₀ and P₁ different from and parallel to each other. Among them, four of the magneto-resistive sensors 110 are arranged in a matrix (2×2) on the reference plane P₀ (a.k.a. a first reference plane), respectively labeled by S₀₀, S₀₁, S₀₂ and S₀₃, and known as first magneto-resistive sensors 1101. Similarly, the other four of the magneto-resistive sensors 110 are arranged in a matrix (2×2) on the reference plane P₁ (a.k.a. a second reference plane), respectively labeled by S₁₀, S₁₁, S₁₂ and S₁₃, and known as second magneto-resistive sensors 1102. Positions of the first magneto-resistive sensors 1101 correspond to positions of the second magneto-resistive sensors 1102, respectively, and their corresponding relationship is, for example, a one-to-one alignment relationship. In addition, in X-axis direction, a spacing between adjacent two of the magneto-resistive sensors 110 is Δx; in Y-axis direction, a spacing between adjacent two of the magneto-resistive sensors 110 is Δy; in Z-axis direction, a spacing between adjacent two of the magneto-resistive sensors 110 is Δz. A midpoint of these magneto-resistive sensors 110 is labeled by O.

The processor 120 is, for example, a device that can perform different operations on signals. In this embodiment, the processor 120 is, for example, a calculator, a micro controller unit (MCU), a central processing unit (CPU) or other programmable microprocessors, a digital signal processor (DSP), a programmable controller, an application specific integrated circuits (ASIC), a programmable logic device (PLD) or other similar hardware, but the invention is not limited thereto. In this embodiment, the processor 120 is coupled to the magneto-resistive sensors 110, and records different position information of the magneto-resistive sensors 110.

A measuring method of the motion sensing device 100 of this embodiment will be described in detail in the following paragraphs.

Referring to FIG. 1, FIG. 2A and FIG. 2B, when the to-be-measured object OB moves, earth magnetic field sensed by the magneto-resistive sensors 110 will change over time, and the processor 120 will determine motion information of the to-be-measured object OB according to the variation of the external magnetic field with respect to time. Here, the motion information is, for example, a velocity of the to-be-measured object OB, but not limited thereto. Next, the processor 120 sequentially performs the following steps.

A step S1: First of all, the processor 120 defines at least one first coordinate system C and at least one corresponding second coordinate system C′ according to the positions of the first and the second magneto-resistive sensors 1101 and 1102. A defining method is as follows: the processor 120 divides the first magneto-resistive sensors 1101 into a first portion P1 and a third portion P3 different from each other, and divides the second magneto-resistive sensors 1102 into a second portion P2 and a fourth portion P4 different from each other. The first portion P1 and the second portion P2 are rotational symmetry to each other, and the third portion P3 and the fourth portion P4 are rotational symmetry to each other. The so-called rotational symmetry means that, when one of the two portions rotated at a certain angle with respect to the midpoint O of the magneto-resistive sensors 110 will overlap with the other, such rotation is called rotational symmetry.

Referring to FIG. 3A, FIG. 3A shows the first coordinate system C₀ and the second coordinate system C₀′ of a first type. In FIG. 3A, the processor 120 makes the first portion P1 include the first magneto-resistive sensors 1101 labeled by S₀₀, S₀₁ and S₀₂, makes the third portion P3 include the first magneto-resistive sensor 1101 labeled by S₀₃, makes the second portion P2 include the second magneto-resistive sensors 1101 labeled by S₁₀, S₁₁ and S₁₂, and makes the fourth portion P4 include the second magneto-resistive sensor 1102 labeled by S₁₃.

Accordingly, the method used by the processor 120 to define the first coordinate system C₀ of FIG. 3A is, for example, using one of the first magneto-resistive sensors 1101 (S₀₀) the first portion P1 as a coordinate origin magneto-resistive sensor CO, and using two of the first magneto-resistive sensors 1101 (S₀₁ and S₀₂) adjacent to the coordinate origin magneto-resistive sensor CO in the first portion P1 as coordinate direction magneto-resistive sensors CD. A vector from the coordinate origin magneto-resistive sensor CO to one of the coordinate direction magneto-resistive sensors 1101 (S₀₁ or S₀₂) is defined as a direction vector of the first coordinate system Co. Also, a vector from the coordinate origin magneto-resistive sensor CO to the second magneto-resistive sensor 1102 (S₁₃) at the corresponding position in the fourth portion P4 is defined as the direction vector of the first coordinate system C₀.

Similarly, the method used by the processor 120 to define the second coordinate system C₀′ of FIG. 3A is, for example, using one of the second magneto-resistive sensors labeled by S₁₀ in the second portion P2 as a coordinate origin magneto-resistive sensor CO′, and using two of the second magneto-resistive sensors 1102 adjacent to the coordinate origin magneto-resistive sensor CO′ and labeled by S₁₁ and S₁₂ in the second portion P2 as coordinate direction magneto-resistive sensors CD′. A vector from the coordinate origin magneto-resistive sensor CO′ to one of the coordinate direction magneto-resistive sensors 1102 (S₁₁ or S₁₂) is defined as a direction vector of the second coordinate system C₀′. Also, a vector from the coordinate origin magneto-resistive sensor CO′ to the first magneto-resistive sensor 1101 (S₀₃) at the corresponding position is defined as the direction vector of the second coordinate system C₀′.

Therefore, the first and the second coordinate systems C₀ and C₀′, can be defined through the above defining process. The first and the second coordinate systems C₀ and C₀′ are also rotational symmetry to each other. The so-called rotational symmetry means that, when one of the two coordinate systems C₀ and C₀′ rotated at a certain angle with respect to the midpoint O of the magneto-resistive sensors 110 will overlap with the other, such rotation is called rotational symmetry.

A step S2: the first magneto-resistive sensors 1101 and the second magneto-resistive sensors 1102 generate a plurality of sensing results according to an external magnetic field, and the processor 120 performs calculations according to the sensing results based on the first and the second coordinate systems C₀ and C₀′ to obtain a calculation result and measures motion information according to the calculation result. Here, the motion information is, for example, a velocity

$${->}_{V}$$

of the to-be-measured object OB. The process of said calculations will be illustrated in the following paragraphs.

In order to explain the calculation process, the following parameters are defined.

$${->}_{V}$$

represents a velocity vector of the to-be-measured object in the three-dimensional space, which may be represented in another manner as (Vₓ, V_{y}, V_{z}), where Vₓ, V_{y}, V_{z} represent velocity components of the to-be-measured object OB in X-axis, Y-axis and Z-axis directions.

$${->}_{S}$$

represents shifts of the magneto-resistive sensors 110 in X-axis, Y-axis and Z-axis directions, which may be represented in another manner as (x, y, z). It is assumed that earth magnetic field is

$${->}_{B} ,$$

which may be represented in another manner as (Bₓ, B_{y}, B_{z}), where Bₓ, B_{y}, B_{z} represent magnetic field components of earth magnetic field in X-axis, Y-axis and Z-axis, respectively.

Therefore, it can be known according to the following equation (1):

$${\to }_{V} = \frac{{d}_{{\to }_{ S }}}{dt} = \frac{{d}_{{\to }_{ S }}}{{d}_{{\to }_{ B }}} \times \frac{{d}_{{\to }_{ B }}}{dt} \qquad ( 1 )$$

wherein

$$\frac{{d}_{{\to }_{ S }}}{dt}$$

represents a differentiation of shift with respect to time,

$$\frac{{d}_{{\to }_{ S }}}{{d}_{{\to }_{ B }}}$$

represents a differentiation of shift with respect to earth magnetic field,

$$\frac{{d}_{{\to }_{ B }}}{dt}$$

represents a differentiation of earth magnetic field with respect to time (also represents the change of earth magnetic field during the movement of the to-be-measured object OB). From perspectives in X-axis, Y-axis and Z-axis directions, equation (1) may be divided into the following three equations (2) to (4):

$$\begin{matrix}{V}_{x} = \frac{dx}{dt} = \frac{dx}{d {B}_{x}} \times \frac{d {B}_{x}}{dt} & ( 2 ) \\ {V}_{y} = \frac{dy}{dt} = \frac{dy}{d {B}_{y}} \times \frac{d {B}_{y}}{dt} & ( 3 ) \\ {V}_{z} = \frac{dz}{dt} = \frac{dz}{d {B}_{z}} \times \frac{d {B}_{z}}{dt} & ( 4 )\end{matrix}$$

which are converted into the form of a vector, that is, the following equation (5):

$${->}_{V} = {J ( {->}_{B} )}^{- 1} \times \frac{d {->}_{B}}{dt} \qquad ( 5 )$$

wherein

$$J {->}_{B}$$

represents a matrix obtained by performing a Jacobian matrix operation on earth magnetic field, and its meaning represents a gradient of earth magnetic field in the three-dimensional space.

$${J ( {->}_{B} )}^{- 1}$$

is an inverse matrix of the matrix obtained by performing the Jacobian matrix operation on earth magnetic field.

Referring to FIG. 4A and FIG. 4B, specifically, it is assumed that a variation in time dt is set to a time difference from tₙ seconds to tₙ₊₁ seconds. At the different times of tₙ seconds and tₙ₊₁ seconds, the first and the magneto-resistive sensors 1101 and 1102 generate the sensing results according to the external magnetic field. S₀₁[x(tₙ)] and S₀₁[x(tₙ₊₁ )] represent the sensing results generated according to the external magnetic field by the first magneto-resistive sensor 1101 labeled by S₀₁ at the times of tₙ seconds and tₙ₊₁ seconds, respectively (i.e., the magnetic field components sensed by the first magneto-resistive sensor 101 labeled by S₀₁ at the times of tₙ seconds and tₙ₊₁ seconds, respectively). The rest may be deduced by analogy. The processor 120 performs the calculation of equation (5) according to the sensing results based on the first and the second coordinate systems C₀ and C₀′. In equation (5), the matrix

$$J {->}_{B}$$

is expanded as shown by FIG. 4A, and the equation of

$$\frac{d {->}_{B}}{dt}$$

is expanded as shown by FIG. 4B.

Referring to FIG. 4A again, for an element at the first row and first column of the matrix

$$J {->}_{B} ,$$

Δx shown by denominator is a spacing between two magneto-resistive sensors in X-axis direction, a result shown by numerator is as shown by the following equation (6):

{S₀₁[x(tₙ₊₁)]−S₀₀[x(tₙ₊₁)]}+{S₀₁[x(tₙ)]−S₀₀[x(tₙ)]}−{S₁₁[x(tₙ₊₁)]−S₁₀[x(tₙ₊₁)]}+{S₁₁[x(tₙ)]−S₁₀[x(tₙ)]} (6)

Next, the above equation (6) is then divided into two equations (7) and (8) as:

{S₀₁[x(tₙ₊₁)]−S₀₀[x(tₙ₊₁)]}+{S₀₁[x(tₙ)]−S₀₀[x(tₙ)]} (7)

{S₁₁[x(tₙ₊₁)]−S₁₀[x(tₙ₊₁)]}+{S₁₁[x(tₙ)]−S₁₀[x(tₙ)]} (8)

In other words, the meaning of the element in the first row and the first column of the above equation (6) is: Equation (7) minus equation (8). Among them, equation (7) represents the meaning of an addition result obtained by adding the sensing results of the two first magneto-resistive sensors 1101 labeled by S₀₁ and S₀₀ in the first coordinate system C₀ at the time of tₙ₊₁ seconds and the time of tₙ seconds; equation (8) represents the meaning of an addition result obtained by adding the sensing results of the two second magneto-resistive sensors 1102 labeled by S₁₀ and S₁₁ in the second coordinate system C₀ at the time of tₙ₊₁ seconds and the time of tₙ seconds. In other words, the element at the first row and the first column represents a difference between the addition results calculated according to the sensing results based on the first and the second coordinate systems C₀ and C₀′.

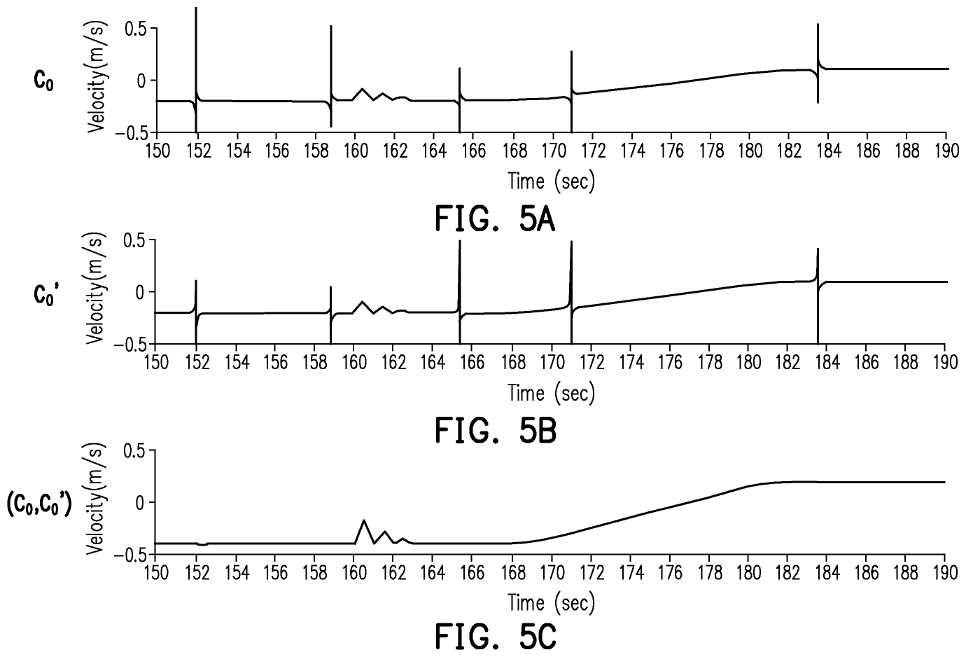

FIG. 5A shows a velocity of the to-be-measured object calculated by the processor according to the sensing results sensed by the magnetoresistive sensors only based on the first coordinate system. FIG. 5B shows a velocity of the to-be-measured object measured by the processor according to the sensing results sensed by the magnetoresistive sensors only based on the second coordinate system. FIG. 5C shows a velocity of the to-be-measured object measured by the processor according to the sensing results sensed by the magnetoresistive sensors based on the first and the second coordinate systems.

Referring to FIG. 5A and FIG. 5B, it can be seen that if the processor 120 measures the velocity of the to-be-measured object OB based on only the first (or the second) coordinate system C₀ (or C₀′), waves (or surges) in opposite directions will be generated before and after certain moments due to the calculations. Specifically, referring to FIG. 5A, before the time of 152 seconds, the calculated velocity goes sharply downward, and after the time of 152 seconds, the calculated velocity goes sharply upward. On the contrary, referring to FIG. 5B, before the time of 152 seconds, the calculated velocity goes sharply upward, and after the time of 152 seconds, the calculated velocity goes sharply downward. The above phenomenon will cause serious errors in calculating the velocity of the to-be-measured object OB.

Referring to FIG. 5C, in the motion sensing device 100 of the present embodiment, because the processor 120 performs the calculations shown by equation (5), FIG. 4A and FIG. 4B according to the sensing results sensed from the external magnetic field by the magneto-resistive sensors 110 based on the first and the second coordinate systems C₀ and C₀′ which are rotational symmetry to each other, the phenomenon of surges can be eliminated by the velocities obtained through in opposite directions before and after certain moments due to the calculations based on the different coordinate systems C₀ and C₀′. In this way, the motion sensing device 100 of this embodiment can accurately measure the motion information of the to-be-measured object OB.

Further, a variation of the external magnetic field is approximately x few or few tens of milligauss (mG), and a size of noise is about the same as its variation. If the variation of the external magnetic field is very small, the conventional technology cannot accurately measure the velocity of the to-be-measured object OB due to noise. On the other hand, the motion sensing device 100 of this embodiment obtains the inverse matrix of the matrix by performing the Jacobian matrix operation according to the sensing results of the magneto-resistive sensors 110 based on the first and the second coordinate systems C₀ and C₀′. In this way, the inverse matrix of the matrix obtained by performing the Jacobian matrix operation can provide the effect of adding and averaging the calculation results of the two coordinate systems C₀ and C₀′. In this process, the effect of noise can be reduced, so the motion sensing device 100 can conduct a accurate measurement.

To further obtain more accurate motion information, after the steps S1 and S2 are performed, the processor 120 can perform the following steps.

A step S3: the processor 120 obtains calculation results corresponding to other first coordinate systems C₁ to C₃ and other second coordinate systems C₁′ to C₃′. Among them, the other first and second coordinate systems C₁ to C₃ and C₁′ to C₃′ are similar to those shown in FIG. 3B to FIG. 3D, and thus related description is omitted hereinafter.

a step S4: at least a portion of all the calculation results (all of them or a portion of them) is obtained and averaged to measure the motion information (the velocity). Accordingly, the motion sensing device 100 can further improve its accuracy.

Moreover, in this embodiment, if the motion sensing device 100 can learn of the velocity of the to-be-measured object OB and an initial position of the to-be-measured object OB according to the above process, the velocity of the to-be-measured object OB may be integrated and then position information of the to-be-measured object OB at a specific time may be obtained according to the initial position of the to-be-measured object OB.

In summary, according to the motion sensing device in the embodiments of the invention, the processor defines the first and the second coordinate systems which are rotational symmetry to each other for the first and the second magneto-resistive sensors disposed on the different reference planes, and performs the calculations according to the sensing results sensed from the external magnetic field by the magneto-resistive sensors based on the first and the second coordinate systems. The directions of the calculation results obtained before and after certain moments will be opposite if only one of the first and the second coordinate system is used. In the embodiments of the invention, by taking both the calculation results of the first and the second coordinate systems into account, the motion sensing device can eliminate the errors derived during the process of the calculations, and thus, can accurately measure the motion information of the to-be-measured object.

1. A motion sensing module suitable for being mounted on a to-be-measured object and used for sensing motion information of the to-be-measured object, the to-be-measured object being placed within a magnetic field range of an external magnetic field, the motion sensing module comprising:
a plurality of first magneto-resistive sensors, disposed on a first reference plane;
a plurality of second magneto-resistive sensors, disposed on a second reference plane, wherein the first reference plane is different from the second reference plane and parallel to the second reference plane, wherein positions of the first magneto-resistive sensors correspond to positions of the second magneto-resistive sensors, respectively; and
a processor, coupled to the first magneto-resistive sensors and the second magneto-resistive sensors, wherein the processor divides the first magneto-resistive sensors into a first portion and a third portion different from each other and divides the second magneto-resistive sensors into a second portion and a fourth portion different from each other,
wherein the processor executes steps of:
a step S1: the processor defines at least one first coordinate system from the first portion of the first magneto-resistive sensors and the second portion of the second magneto-resistive sensors, and the processor defines at least one second coordinate system from the third portion of the first magneto-resistive sensors and the fourth portion of the second magneto-resistive sensors, wherein the first coordinate system and the second coordinate system are rotational symmetry to each other; and
a step S2: the first magneto-resistive sensors and the second magneto-resistive sensors generate a plurality of sensing results according to an external magnetic field, and the processor performs calculations according to the sensing results based on the first coordinate system and the second coordinate system to obtain a calculation result and measures motion information according to the calculation result.

2. The motion sensing module of claim 1, wherein the processor further executes steps of:
a step S3: repeating the step S1 and the step S2 to obtain calculation results corresponding to other first coordinate systems and other second coordinate systems; and
a step S4: obtaining and averaging at least a portion of all the calculation results to measure the motion information.

3. The motion sensing module of claim 1, wherein the motion information is a velocity of the to-be-measured object.

4. The motion sensing module of claim 3, wherein in the step S2, the processor performs the calculations according to the sensing results based on the first coordinate system and the second coordinate system to measure the velocity of the to-be-measured object by an equation:
$${->}_{V} = {J ( {->}_{B} )}^{- 1} \times \frac{d {->}_{B}}{dt}$$
wherein
$${->}_{V}$$
is the velocity of the to-be-measured object,
$${J ( {->}_{B} )}^{- 1}$$
an inverse matrix of a matrix obtained by the processor after performing a Jacobian matrix operation according to the sensing results based on the first coordinate system and the second coordinate system, and
$$\frac{d {->}_{B}}{dt}$$
is a differential operation of the sensing results with respect to time.

5. The motion sensing module of claim 3, wherein after integrating the velocity of the to-be-measured object with respect to time, the processor obtains position information of the to-be-measured object at a specific time according to an initial position of the to-be-measured object.

6. The motion sensing module of claim 1, wherein
the processor uses one of the first magneto-resistive sensors in the first portion as a coordinate origin magneto-resistive sensor, and uses two of the first magneto-resistive sensors adjacent to the coordinate origin magneto-resistive sensor in the first portion and one of the second magneto-resistive sensors corresponding to the coordinate origin magneto-resistive sensor as coordinate direction magneto-resistive sensors,
wherein a vector from the coordinate origin magneto-resistive sensor to one of the coordinate direction magneto-resistive sensors is defined as a direction vector of the first coordinate system.

7. The motion sensing module of claim 1, wherein
the processor uses one of the second magneto-resistive sensors in the second portion as a coordinate origin magneto-resistive sensor, and uses two of the second magneto-resistive sensors adjacent to the coordinate origin magneto-resistive sensor in the second portion and one of the first magneto-resistive sensors corresponding to the coordinate origin magneto-resistive sensor as coordinate direction magneto-resistive sensors,
wherein a vector from the coordinate origin magneto-resistive sensor to one of the coordinate direction magneto-resistive sensors is defined as a direction vector of the second coordinate system.

8. The motion sensing module of claim 1, wherein the positions of the first magneto-resistive sensors are aligned with the positions of the second magneto-resistive sensors in a one to one manner.

9. The motion sensing module of claim 1, wherein the first portion and the second portion are rotational symmetry to each other, and the third portion and the fourth portion are rotational symmetry to each other.
